(19) United States
(12) Patent Application Publication
(10) Pub. No.: US 2025/0367472 A1
(43) Pub. Date: Dec. 4, 2025

(54) METHOD AND DEVICE FOR GENERATING TREATMENT PLAN, AND MEDIUM

(71) Applicant: OUR UNITED CORPORATION, Xi'an (CN)

(72) Inventors: Peng GUO, Xi'an (CN); Jinsheng LI, Xi'an (CN)

(21) Appl. No.: 18/852,081

(22) PCT Filed: Mar. 29, 2022
(86) PCT No.: PCT/CN2022/083824

§ 371 Date: Sep. 27, 2024

(51) Int. Cl. A61N 5/10; G16H 20/10

(52) U.S. Cl. CPC A61N 5/1039; G16H 20/10

(57) ABSTRACT

The embodiments of the present application relate to the technical field of medical information. Provided are a treatment plan generation method and apparatus, and a storage medium. The method comprises: acquiring an objective contour of an objective target region; searching a preset target mapping relationship for an objective target set which corresponds to the objective contour, wherein the objective target set comprises the number of targets and the size of each target; determining the position of each target in the objective target region based on the size of each target; determining the position of each target in the objective target region according to the size of each target; and determining the dose of each target according to the position of each target and a preset prescribed dose, and generating a treatment plan. The present application can improve the formulation efficiency of a treatment plan.

### TECHNICAL FIELD

The present disclosure relates to the field of medical information technologies, and in particular, to a method and apparatus for generating a treatment plan, a device, and a medium.

### BACKGROUND

Radiation therapy, also referred to as radiotherapy, is a common cancer treatment method. Before radiotherapy is applied to a patient by radiotherapy equipment, it is necessary to design a treatment plan for the patient.

Currently, treatment plans are mostly acquired by designing, by physicists based on their clinical experience, the number, sizes, and positions of targets within a target volume, the dose of each of the targets, and the like using a treatment plan system (TPS).

However, the artificial design of the treatment plan requires a high level of clinical experience of the physicists, and to ensure that the designed treatment plans meet the prescribed doses, a large number of trial-and-error adjustments are inevitable, which results in a long time consumed by the whole process.

### SUMMARY

Embodiments of the present disclosure provide a method and apparatus for generating a treatment plan, a device, and a medium, to improve the generation efficiency of treatment plans.

In a first aspect, the embodiments of the present disclosure provide a method for generating a treatment plan. The method includes:
acquiring a designated contour of a designated target volume;
searching, in a predetermined target mapping relationship, a designated target set corresponding to the designated contour, the designated target set comprising a total number of targets and a size of each of the targets;
determining a position of each of the targets within the designated target volume based on the size of each of the targets; and
determining a dose of each of the targets based on the position of each of the targets and a predetermined prescription dose, and generating a treatment plan.

In some embodiments, determining the position of each of the targets within the designated target volume based on the size of each of the targets includes:
determining a mask of each of the targets based on the size of each of the targets; and
determining the position of each of the targets within the designated target volume by performing convolutional shape matching between the mask and the designated contour.

In some embodiments, determining the dose of each of the targets based on the position of each of the targets and the predetermined prescription dose includes:
acquiring a dose curve distribution in the designated target volume by performing a dose calculation based on the size, the position, and a weight of each of the targets; and
determining the dose of each of the targets based on the dose curve distribution and the predetermined prescription dose.

In some embodiments, prior to searching, in the predetermined target mapping relationship, the designated target set corresponding to the designated contour, the method further includes:
acquiring a plurality of target volumes;
acquiring contours of the plurality of target volumes by delineating the plurality of target volumes;
acquiring target sets corresponding to the contours by deep reinforcement learning and training based on each of the contours; and
establishing the predetermined target mapping relationship based on the contours and the target sets corresponding to the contours.

In some embodiments, acquiring the target sets corresponding to the contours by deep reinforcement learning and training based on each of the contours includes:
forming, based on each of the contours, a mask of a target volume corresponding to the contour;
constructing a state matrix corresponding to the a target volume based on the mask, wherein the state matrix includes the mask; and
acquiring a target set within the contour based on the state matrix.

In some embodiments, acquiring the target set within the corresponding contour based on the state matrix includes:
determining and acquiring a size of a first target by performing feature extraction on the state matrix based on a convolutional neural network;
determining a position of the first target and a dose of the first target based on the size of the first target, and updating the state matrix corresponding to the target volume;
determining sizes, positions, and doses of subsequent targets sequentially based on the updated state matrix until the predetermined prescription dose is satisfied, and updating the state matrix corresponding to the target volume; and
counting a number of the targets and determining the number of the targets and the sizes of the targets as the target set corresponding to the contour.

In some embodiments, acquiring the size of the first target by performing feature extraction on the state matrix based on the convolutional neural network includes:
acquiring an initial state feature corresponding to the target volume by performing feature extraction on the state matrix using the convolutional neural network; and
acquiring the size of the first target by processing the initial state feature using a predetermined action selection network.

In some embodiments, determining the position of the first target and the dose of the first target based on the size of the first target includes:
determining a mask of the first target based on the size of the first target;
determining the position of the first target by performing convolutional shape matching between the contour and the mask of the first target; and
determining the dose of the first target based on the position of the first target.

In some embodiments, the state matrix further includes a dose state corresponding to the target volume, and updating the state matrix corresponding to the target volume includes:
calculating dose state information corresponding to t targets within the target volume based on a dose of a t^{th} target and a size of the target volume, wherein t is an integer greater than or equal to 1; and
updating the dose state based on the dose state information of the t targets.

In some embodiments, the dose state includes a dose coverage distribution, a dose conformity distribution, and a dose overflow distribution; and the dose state information includes dose coverage information, dose conformity information, and dose overflow information; and
updating the dose state based on the dose state information of the t targets includes:
updating the dose coverage distribution, the dose conformity distribution, and the dose overflow distribution respectively based on the dose coverage information, dose conformity information, and dose overflow information of the t targets.

In some embodiments, the method further includes:
calculating reward information of the t^{th} target based on the dose coverage information, dose conformity information, and dose overflow information of the t targets.

In some embodiments, the method further includes:
calculating current cumulative reward information corresponding to the t targets within the target volume based on the reward information of the t^{th} target;
wherein the current cumulative reward information indicates a reliability of a current target set within the contour.

In some embodiments, the method further includes:
storing the updated state matrix, the current target set within the target volume, and the current cumulative reward information.

In some embodiments, the method further includes:
calculating a relative advantage parameter between the current target set within the contour and a previous target set within the contour based on the current cumulative reward information and history cumulative reward information corresponding to the target volume, wherein the history cumulative reward information indicates a reliability of the previous target set; and
determining and updating a designated target set within the contour from the current target set and the previous target set based on the relative advantage parameter.

In a second aspect, the embodiments of the present disclosure further provide an apparatus for generating a treatment plan. The apparatus includes:
an acquiring module, configured to acquire a designated contour of a designated target volume;
a searching module, configured to search, in a predetermined target mapping relationship, a designated target set corresponding to the designated contour, the designated target set comprising a total number of targets and a size of each of the targets;
a first determining module, configured to determine a position of each of the targets within the designated target volume based on the size of each of the targets; and
a second determining module, configured to determine a dose of each of the targets based on the position of each of the targets and a predetermined prescription dose, and generate a treatment plan.

In a third aspect, the embodiments of the present disclosure further provide a computer device. The computer device includes a memory and a processor, wherein the memory stores one or more computer programs executable by the processor, and the processor, when loading and executing the one or more computer programs, is caused to perform the method for generating a treatment plan described in above first aspect.

In a fourth aspect, the embodiments of the present disclosure further provide a non-transitory storage medium. The storage medium stores one or more computer programs, wherein the one or more computer programs, when read and executed by a processor of a device, cause the device to perform the method for generating a treatment plan described in the above first aspects.

According to the method and apparatus for generating a treatment plan, the device, and the medium provided by the embodiments of the present disclosure, a designated target set within a designated contour of a designated target volume is determined by performing shape matching with a pre-acquired target mapping relationship based on the designated contour of the designated target volume, which achieves the determination of an optimal target combination within the designated target volume. In the case that the optimal target combination, i.e., the designated target set is determined, the position of each of the targets within the designated target volume is determined based on the size of each of the targets in the determined designated target set, that is, the optimal position of each size of target within the designated target volume is determined. Subsequently, the dose of each target is determined based on the position of each target and the predetermined prescription dose, which achieves the dose calculation of each target at the corresponding position within the designated target volume. In this way, the total number of targets, the optimal target combination of various target sizes, positions of the targets, and doses of the targets are automatically and programmatically calculated during the process of generating the treatment plan, which avoids the repetition of various steps in the artificial design process of treatment plans, simplifies the process of making and generating the treatment plan, reduces the dependence of the treatment plan on clinical experience, improves the precision and design efficiency of the treatment plan, and thus effectively guarantees the application of the treatment plan.

### BRIEF DESCRIPTION OF DRAWINGS

To describe the technical solutions in the embodiments of the present disclosure more clearly, the following briefly introduces the accompanying drawings required for describing the embodiments. It is understood that the accompanying drawings in the following description show merely some embodiments of the present disclosure and should not be considered as a limit to the scope of the present disclosure, and a person of ordinary skill in the art may still derive other drawings from these accompanying drawings without creative efforts.

FIG. 1 is a flowchart of a method for generating a treatment plan according to some embodiments of the present disclosure;

FIG. 2 is a flowchart of a method of determining a dose of a target in a method for generating a treatment plan according to some embodiments of the present disclosure;

FIG. 3 is a flowchart of a method of training a predetermined target mapping relationship in a method for generating a treatment plan according to some embodiments of the present disclosure;

FIG. 4 is a flowchart of a method of deep reinforcement learning and training of a target volume contour in a method for generating a treatment plan according to some embodiments of the present disclosure;

FIG. 5 is a flowchart of a method of acquiring a target set within a target volume in a method for generating a treatment plan according to some embodiments of the present disclosure;

FIG. 6 is a schematic diagram of a framework of a training module in a method for generating a treatment plan according to some embodiments of the present disclosure;

FIG. 7 is a flowchart of yet another method of deep reinforcement learning and training of a target volume contour in a method for generating a treatment method according to some embodiments of the present disclosure;

FIG. 8 is a schematic diagram of an apparatus for generating a treatment plan according to some embodiments of the present disclosure; and

FIG. 9 is a schematic diagram of a computer device according to some embodiments of the present disclosure.

### DETAILED DESCRIPTION

In order to make the objects, technical solutions, and advantages of the embodiments of the present disclosure clearer, the technical solutions in the embodiments of the present disclosure are described clearly and completely hereinafter in conjunction with the accompanying drawings in the embodiments of the present disclosure, and it is clear that the described embodiments are merely some embodiments of the present disclosure rather than all of the embodiments.

In contrast to the manner of artificially designing a treatment plan according to conventional technology, the embodiments of the present disclosure are intended to provide a method in which a treatment plan for a designated target volume is generated automatically through an algorithmic routine without human intervention, which avoids repeated trial-and-error adjustments in the process of designing the treatment plan and improves the generation efficiency of the treatment plan.

In some embodiments, the device configured for performing the method for generating a treatment plan provided in the embodiments of the present disclosure is a computer device installed with a treatment plan generation algorithm, and the computer device performs a corresponding method for generating a treatment plan by running the treatment plan generation algorithm. In some embodiments, the treatment plan generation algorithm is a sub-function module of TPS, which is also referred to as a TPS calculation module. It is to be noted that the treatment plan generated by the method for generating a treatment plan provided by the embodiments of the present disclosure is applicable to any radiation therapy device. In some embodiments, the radiation therapy device includes a focused therapy device or a conformal therapy device. The following embodiments are illustrated by taking a multi-source focused treatment head, specifically a gamma knife treatment head as an example.

Gamma Knife is a common cancer treatment device. Currently, Gamma Knife treatment plans are acquired mostly by designing, by physicists using a treatment planning system (TPS) based on clinical experience, numbers of targets with different sizes and positions of targets, to ensure that the target volume meets the prescribed dose and at the same time the organ at risk (OAR) is irradiated as little as possible, which is a process including a large number of trial-and-error adjustments, therefore the process is complicated and time-consuming.

Existing Gamma Knife treatment plan optimization methods are mainly based on geometric features, that is, combinations of the number of targets of different sizes need to be estimated based on the shape and area of the target volume, and the process of plan optimization is a process of optimizing the positions of the targets. The artificially designed treatment plan is able to ensure that the prescribed dose is met but is time-consuming. The automatic design for a treatment plan based on the optimization of the target position is fast and needs less human labor, but the initialization of the combination of target sizes requires a high level of experience, the optimization process is lengthy, the results are often unsatisfactory, and a physicist is required to make adjustments in the end.

To address the above problems, the embodiments of the present disclosure provide a treatment plan algorithm based on deep reinforcement learning and shape matching. Deep reinforcement learning is an artificial intelligence algorithm with an image as input that combines the perception ability of deep learning for environmental features and the decision-making ability of reinforcement learning for events. In deep reinforcement learning, the agent, during interactions with the environment (Env), acquires an image state from the environment at a moment t, performs some kind of action based on a current state, and acquires a corresponding reward or penalty. The purpose of the reinforcement learning algorithm is to learn a set of action policies by training to maximize a future cumulative reward. Because the reinforcement learning and training process needs no human experience data as training templates, the human labor cost is greatly reduced. Therefore, the deep reinforcement learning algorithm is suitable for Gamma Knife treatment plan design, which allows computers to repeat the trial-and-error process to acquire the optimal treatment plan, reduces the reliance on clinical experience, eliminates the need to artificially set the target combinations and target positions, and eliminates the need for artificial training samples, therefore improves the effectiveness of the treatment plan and enhances the efficiency of the physicist in making the treatment plan.

A method for generating a treatment plan provided in the embodiments of the present disclosure is first illustrated in combination with a plurality of exemplary embodiments hereafter. FIG. 1 is a flowchart of the method for generating a treatment plan according to some embodiments of the present disclosure. As shown in FIG. 1, the method for generating a treatment plan includes the following processes in some embodiments.

In process S101, a designated contour of a designated target volume is acquired.

In some embodiments, an image of the object to be irradiated is acquired in advance, and a contour of the image is delineated to acquire a contour of a designated target volume of the object to be irradiated in the image, which is also known as a designated contour. The designated target volume includes a region to be irradiated of the object to be irradiated. In some embodiments, the object to be irradiated includes a phantom, a human body, or an animal. In some embodiments, the designated target volume is a tumor region of the human body or a tumor region of the animal, etc., and the designated contour is the contour of the tumor region. The contour is manually delineated by the physician or automatically delineated. In the embodiments of the present disclosure, the designated contour of the designated target volume that is pre-drew is acquired from a predetermined storage location, or the designated contour of the designated target volume that is delineated in real-time is acquired from a target volume contour device.

It should be noted that the designated target volume is also referred to as a planning target volume (PTV), and the designated contour is also referred to as a PTV shape or a PTV contour.

In process S102, a designated target set corresponding to the designated contour is searched in a predetermined target mapping relationship.

The designated target set includes a total number of targets and a size of each of the targets. At least target sets of a plurality of target volume contours are stored in the predetermined target mapping relationship. In some embodiments, the designated contour is shape-matched with the various target volume contours in the predetermined target mapping relationship, and based on the shape-matching results, a target set within a target volume contour in the predetermined target mapping relationship that has the highest shape-matching degree with the designated target volume is determined as the designated target set corresponding to the designated contour. That is, corresponding to the designated contour means that the shape of a target contour matches the shape of the designated contour. The target set within each of the target volume contours in the predetermined target mapping relationship includes a total number of targets within a corresponding target volume contour, and a size of each of the targets, which refers to the size of each of the targets, that are optimally placed within the corresponding target volume contour, and the number of targets with different sizes in some embodiments. In other words, in the predetermined target mapping relationship, the target set within each target volume contour is a combination of the number of targets with different sizes within the corresponding target volume contour, i.e., an optimal combination of the target sizes and target numbers with the corresponding target volume contour. The predetermined target mapping relationship is stored in advance in a memory unit in some embodiments, and the designated target set searched from the predetermined target mapping relationship stored in the memory unit is the optimal target strategy for the designated target volume.

In the following, unless otherwise specified, for ease of description, the number of targets and the sizes of the targets involved as followed refer to the total number of targets and the sizes of the targets in the designated target set.

In process S103, a position of each of the targets within the designated target volume is determined based on the size of each of the targets.

In some embodiments, based on the size of each of the targets, the optimal position of the target of the corresponding size placed within the designated target volume (i.e., the position of each of the targets within the designated target volume) is determined by performing shape-matching with the designated contour of the designated target volume.

In some embodiments, determining the position of each of the targets within the designated target volume based on the size of each of the targets in process S103 includes:
determining a mask of each of the targets based on the size of each of the targets; and
determining the position of each of the targets within the designated target volume by performing convolutional shape matching between the mask and the designated contour.

In some embodiments, a mask of a target is generated using a predetermined target mask generation method based on the size of the target and a predetermined shape of the target, and the target mask is a target mask corresponding to the size of the target, one size of the targets corresponding to one target mask, and different sizes corresponding to different target masks. Upon acquiring the target mask, the optimal position of the target of the size to be placed within the designated target volume (i.e., the position of the target within the designated target volume) is determined by performing convolutional shape matching between the target mask and the designated contour. In some embodiments, the target mask and the designated contour are input into a predetermined convolutional shape-matching network to achieve the convolutional shape matching between the target mask and the designated contour through the convolutional shape-matching network, to acquire the position of the target within the designated target volume.

In process S104, a dose of each of the targets is determined based on the position of each of the targets and a predetermined prescription dose, and a treatment plan is generated.

In some embodiments, each of the targets is placed at a corresponding position within the designated target volume, and the dose of each of the targets placed at the corresponding position (i.e., the dose of each of the targets) is calculated in combination with the predetermined prescription dose, wherein the predetermined prescription dose refers to a predetermined prescription dose for the designated target volume. In some embodiments, in the process of calculating the dose of each of the targets, each of the targets is placed at a corresponding position within the designated target volume, a dose calculation is carried out for each of the targets to acquire a dose distribution of each of the targets at the corresponding position, and then the dose of each of the targets is calculated based on the dose distribution as well as the predetermined prescription dose.

Making a treatment plan itself means determining the number of targets within the designated target volume, the size of each of the targets, and the dose for each of the targets for radiotherapy within a specific designated target volume. Therefore, upon acquiring the dose for each of the targets, a treatment plan for the designated target volume is generated based on the number of targets in the designated target set, the size of each of the targets, the position of each of the targets, and the dose of each of the targets.

According to the method for generating a treatment plan provided in embodiments of the present disclosure, a designated target set within a designated contour of a designated target volume is determined by performing shape matching with a pre-acquired target mapping relationship based on the designated contour of the designated target volume, which achieves the determination of an optimal target combination within the designated target volume. In the case that the optimal target combination, i.e., the designated target set is determined, the position of each of the targets within the designated target volume is determined based on the size of each of the targets in the determined designated target set, that is, the optimal position of each size of target within the designated target volume is determined. Subsequently, the dose of each target is determined based on the position of each target and the predetermined prescription dose, which achieves the dose calculation of each target at the corresponding position within the designated target volume. In this way, the number of targets, the optimal target combination of various target sizes, positions of the targets, and doses of the targets are automatically and programmatically calculated during the process of generating the treatment plan, which avoids the repetition of various steps in the artificial design process of treatment plans, simplifies the process of making and generating the treatment plan, reduces the dependence of the treatment plan on clinical experience, improves the precision and design efficiency of the treatment plan, and thus effectively guarantees the application of the treatment plan.

In addition, in the method provided by the present embodiments, the mask of each of the targets is determined based on the size of the target, and the position of each of the targets in the designated target volume is determined by performing convolutional shape matching between the mask and the designated contour, which achieves automatic determination of the optimal positions of the targets of different sizes, avoids manual and repeated determination and adjustment of the position of the target, and improves the precision as well as the generation efficiency of the treatment plan.

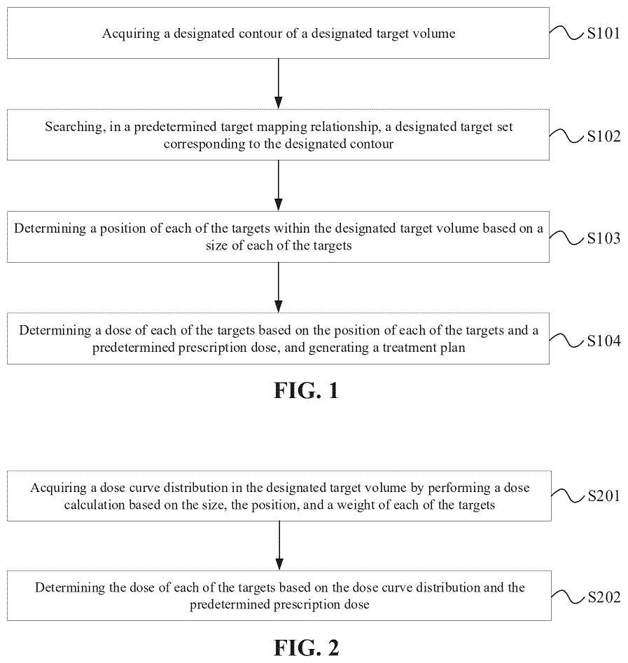

On the basis of the method for generating a treatment plan provided by the above embodiments, with respect to the implementation of determining the dose of each target mentioned in the above embodiments, the embodiments of the present disclosure provide an optional method. FIG. 2 is a flowchart of a method for determining a dose of a target in the method for generating a treatment plan according to the embodiments of the present disclosure. As shown in FIG. 2, in some embodiments, determining the dose of each of the targets based on the position of each of the targets and the predetermined prescription dose in process S104 of the previous method includes the following processes.

In process S201, a dose curve distribution of the designated target volume is acquired by performing a dose calculation based on the size, the position, and a weight of each of the targets.

In some embodiments, the dose curve distribution of the designated target volume with the targets placed at corresponding positions within the designated target volume is calculated using a predetermined dose calculation method based on the size and position of each of the targets and the weight of each of the targets within the designated target volume. In some embodiments, the predetermined dose calculation method is a Monte Carlo simulation calculation method or other dose calculation method. In some embodiments, the dose curve distribution is a 50% dose curve distribution, that is, a region within the designated target volume that is defined by a 50% dose line; or the dose curve distribution is any of other percentage dose lines, which can be set according to actual needs and is not limited in the embodiments of the present disclosure.

In process S202, the dose of each of the targets is determined based on the dose curve distribution and the predetermined prescription dose.

The dose curve distribution of the designated target volume with each of the targets placed at a corresponding position within the designated target volume is acquired by performing the dose calculation, so that the dose of each of the targets is acquired by multiplying the dose curve distribution with the predetermined prescription dose. In some embodiments, upon multiplying the dose curve distribution with the predetermined prescription dose, the calculated dose is corrected or adjusted using other target dose correction methods, which is not limited in the embodiments of the present disclosure.

For a better understanding of the present disclosure, the dose curve distribution and the dose are illustrated. In some embodiments, the dose curve distribution acquired by dose calculation is configured to characterize the dose distribution within the designated target volume, but is not equal to the actual dose, and the dose of each of the targets determined based on the predetermined prescription dose is the actual dose of each of the targets, which is used to guide the dose of radiation at the target based on the treatment plan.

In the method provided by the present embodiments, the dose calculation is first performed based on the size, the position, and a weight of each of the targets to acquire a dose curve distribution in the designated target volume, and then the dose of each of the targets is determined based on the dose curve distribution and the predetermined prescription dose, which achieves the calculation of the dose of each of the targets within the designated target volume, ensures the prescription dose requirement within the designated target volume being met, avoids repeated confirmation for the dose of the target during the artificial design of the treatment plan, reduces the time consumed for ensuring the prescription dose during the generation of the treatment plan.

In some embodiments, the predetermined target mapping relationship as shown above is acquired in advance by deep reinforcement learning and training based on a plurality of target volume contours. In some embodiments, acquiring the predetermined target mapping relationship by deep reinforcement learning and training is performed by a processing unit of a computer device, which is a central processing unit or a graphics processing unit (GPU) in some embodiments. Using the GPU to perform deep reinforcement learning and training ensures the training efficiency of the predetermined target mapping relationship. The computer device that performs the deep reinforcement learning and training to acquire the predetermined target mapping relationship and the computer device that performs the method for generating a treatment plan are the same computer device or different computer devices.

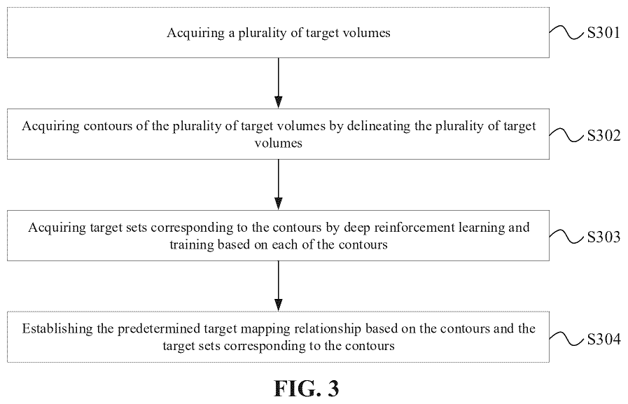

The following are illustrated in conjunction with specific examples. FIG. 3 is a flowchart of a method for training a predetermined target mapping relationship in a method for generating a treatment plan according to some embodiments of the present disclosure. As shown in FIG. 3, prior to searching, in the predetermined target mapping relationship, the designated target set corresponding to the designated target contour in the process S102 of the method described above, the method further includes the following processes in some embodiments.

In process S301, a plurality of target volumes are acquired.

In some embodiments, the plurality of target volumes are a plurality of target volumes with random shapes. The plurality of target volumes are generated simultaneously or sequentially, which is not limited in the embodiments of the present disclosure. The plurality of target volumes are target volumes acquired by acquiring a plurality of medical images of different target volumes, and then extracting the target volume image therefrom; or the plurality of target volumes are a plurality of target volumes of different shapes generated by simulation.

In process S302, contours of the plurality of target volumes are acquired by delineating the plurality of target volumes.

The delineating of each target volume is similar to the implementation of acquiring the target volume contour of the designated target volume in the above embodiments. More details can refer to the above embodiments and are not repeated herein.

In process S303, target sets corresponding to the contours are acquired by deep reinforcement learning and training based on each of the contours.

In some embodiments, a predetermined training module is used to train a planning model that satisfies the prescription dose requirement of each target volume contour, i.e., the target sets corresponding to the target volume contours, by deep reinforcement learning and training based on each of the target volume contours. During the process of training each target volume contour by deep reinforcement learning, the training is carried out continuously by way of machine trial and error for each target volume contour until the number of targets permitted to be placed within the corresponding target volume contour and the size of each of the targets are determined in the case of satisfying the prescription dose requirement, and in this way, the target sets corresponding to the target volume contours are acquired.

In process S304, a predetermined target mapping relationship is established based on the contours and the target sets corresponding to the contours.

In some embodiments, upon acquiring the target set within each contour by performing deep reinforcement learning and training on the contours of the plurality of target volumes, a mapping relationship between the contours and the target sets is established based on the contours and the target sets corresponding to the contours, so as to achieve the establishment of the predetermined target mapping relationship. In some embodiments, in the case that the predetermined target mapping relationship is established, the predetermined target mapping relationship is stored in a memory unit as an empirical parameter acquired by deep reinforcement learning and training. The memory unit is also referred to as an experience memory unit.

According to the method provided by the embodiments of the present disclosure, during the process of deep reinforcement learning and training for each target volume contour, the target sets corresponding to the target volume contours are acquired by repeated trial and error by the machine, such that the optimal target combination is acquired, which achieves automatic generation of the target contours corresponding to the target volume contours without manually setting the target combination and without requiring human empirical data as a training sample. In this way, the cost of manpower and the dependence on clinical experience are greatly reduced, and thus the effectiveness of the treatment plan generated based on the target set is effectively ensured, and the generation efficiency of the treatment plan by the physicist is improved.

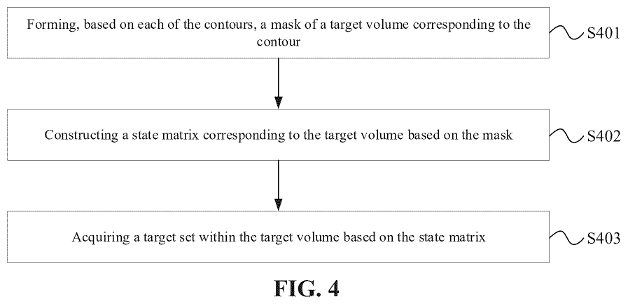

The deep reinforcement learning and training process for each target volume contour is explained and illustrated by specific embodiments in conjunction with the accompanying drawings hereafter. FIG. 4 is a flowchart of a method of deep reinforcement learning and training on a target volume contour in the method for generating a treatment plan according to some embodiments of the present disclosure. In some embodiments, as shown in FIG. 4, acquiring the target sets corresponding to the target volume contours by deep reinforcement learning and training based on each of the target volume contours in process S303 of the method as illustrated above includes the following processes.

In process S401, based on each of the contours, a mask of a target volume corresponding to the contour is formed.

In some embodiments, a target volume mask is formed by processing a predetermined three-dimensional image based on the target volume contour and the organ at risk (OAR) image. The organ at risk image refers to an image containing a normal organ, i.e., an organ that has not undergone a lesion, that is located around the region corresponding to the target volume contour. In some embodiments, the predetermined three-dimensional image is a three-dimensional image having a predetermined size, or is called a three-dimensional matrix of uniform size. In some embodiments, in the process of forming the target volume mask, different operations are performed respectively on the region corresponding to the target volume contour in the predetermined three-dimensional image and the OAR region based on the target volume contour to generate the target volume mask. In this way, the region corresponding to the target volume contour in the target volume mask is differentiated from the OAR region in the target volume mask, and at the same time, the space for subsequently searching for positions of targets is restricted to the region corresponding to the target volume contour in the target volume mask, so as to avoid the target position falls into the OAR region, thus avoiding therapeutic damage to the OAR region.

In some embodiments, in order to unify the search space for the position of the target volume, in the process of forming the target volume mask, the pixel values of a region corresponding to the target volume contour in the three-dimensional pixel matrix of the predetermined size are set to 1, the pixel values of the OAR region are set to −1, and the pixel values of the other tissue regions are set to 0. That is, the generated target volume contour is actually a three-dimensional pixel matrix, i.e., a three-dimensional image of the target volume mask.

Although the search space of the target position is in the three-dimensional pixel matrix of the target volume mask, due to the different pixel values of different regions in the target volume mask, in the process of acquiring the target set based on the subsequently generated state matrix of the target volume mask, the target position within the target volume corresponding to the target volume contour is selected by only taking the region with the pixel value of 1 as an available domain, i.e., the target position is selected based on the region corresponding to the target volume contour in the target volume mask.

In process S402, a state matrix corresponding to the target volume is constructed based on the mask.

In some embodiments, the state matrix corresponding to the target volume is constructed based on the mask using a predetermined state matrix model. The state matrix model, as an environment state matrix of the deep reinforcement learning system, is configured to characterize the environment state features, which includes a multi-layer feature matrix, in some embodiments, with the target volume mask as one of the layers to characterize the state features of the target volume mask. That is, the state matrix is at least configured to characterize a state matrix of the target volume mask. In some embodiments, the target volume mask is configured as a first layer of the feature matrix in the state matrix, i.e., a base feature matrix corresponding to the target volume.

In process S403, a target set within the contour is acquired based on the state matrix.

In some embodiments, deep reinforcement learning and training are performed on the target volume contour based on the state matrix until a target set within the target volume contour satisfying the predetermined prescription dose requirement is acquired.

According to the method provided by the present embodiments, on the basis of acquiring the target volume mask of the target volume corresponding to the target volume contour based on the target volume contour, the targets are restricted in a region corresponding to the target volume contour for the target volume with a specific shape, i.e., the target volume contour, so as to avoid searching for the target positions in other regions, reduce the search space for the target positions, and improve the training speed based on the target volume contour, thereby effectively improving the generation speed of the treatment plan.

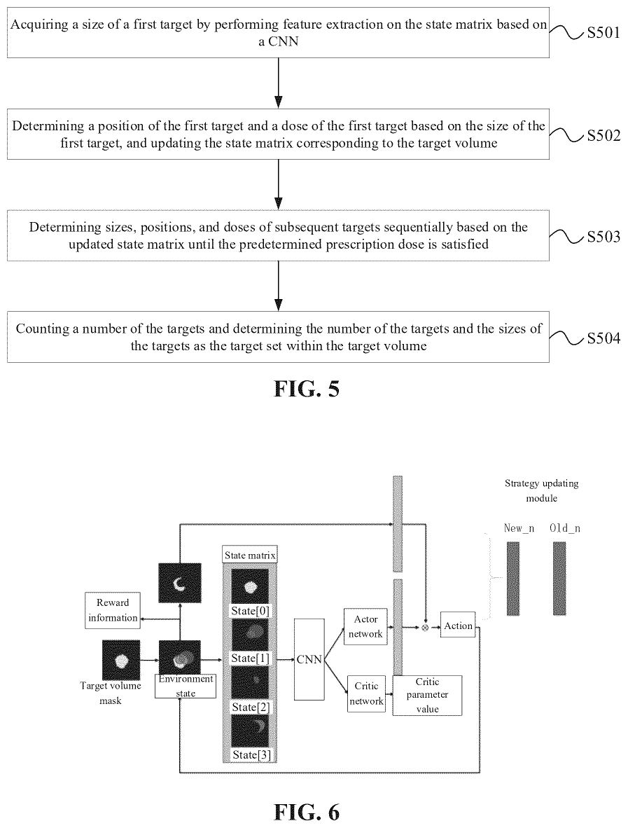

On the basis of the deep reinforcement learning and training shown in FIG. 4 above, the embodiments of the present disclosure further provide an optional implementation of acquiring a target set based on a state matrix. FIG. 5 is a flowchart of a method for acquiring a target set within a target volume contour in a method for generating a treatment plan according to some embodiments of the present disclosure. In some embodiments, as shown in FIG. 5, acquiring the target set within the target volume contour based on the state matrix in process S403 of the above method includes the following processes.

In process S501, a size of a first target is acquired by performing feature extraction on the state matrix based on a convolutional neural network (CNN).

In some embodiments, an initial state feature corresponding to the target volume is acquired by performing feature extraction on the state matrix using a convolutional neural network; and the size of the first target is acquired by processing the initial state feature using a predetermined action selection network.

In some embodiments, during the learning process based on the state matrix, the state matrix is input to a predetermined agent, which is also known as an intelligent network model, to acquire the size of the first target. In some embodiments, the predetermined agent includes a CNN, feature extraction is performed on the state matrix based on the CNN to acquire the initial state feature corresponding to the target volume. Upon acquiring the initial state feature, the size of the first target is acquired by simulating a process of placing the first target within the corresponding target volume based on the initial state feature.

In some embodiments, the predetermined agent further includes an action selection network, which is also known as an actor network (ActorNet). An input layer of the action selection network is connected to an output layer of the CNN, such that the initial state feature corresponding to the target volume acquired by the CNN is input into the action selection network. In this way, upon acquiring the initial state feature corresponding to the target volume by performing feature extraction on the state matrix using the CNN, the size of the first target is acquired by processing the initial state feature using the action selection network.

In some embodiments, the CNN is a three-layer three-dimensional convolutional neural network, and the action selection network is an intelligent network model of a neural network architecture to determine the size of the target based on the state feature extracted by the CNN. In some embodiments, the action selection network is a neural network model based on a proximal policy optimization (PPO) algorithm, which acquires the target size using the PPO algorithm based on the extracted state feature. In some embodiments, the action selection network acquires the size of the first target using the PPO algorithm based on the initial state feature.

In process S502, a position of the first target and a dose of the first target are determined based on the size of the first target, and the state matrix corresponding to the target volume is updated.

In some embodiments, in the case that the size of the first target is determined, the size of the first target and the contour of the target volume are shape-matched to determine the optimal position of the first target with the corresponding size to be placed within the designated target volume, i.e., the position of the first target, and then the dose of the first target is determined based on the position of the first target and the predetermined prescription dose.

In some embodiments, determining the position of the first target and the dose of the first target based on the size of the first target in the above process S103 includes:
determining a mask of the first target based on the size of the first target;
determining the position of the first target by performing convolutional shape matching between the mask of the first target and the target volume contour; and
determining a dose of the first target based on the position of the first target.

In this embodiment, the detailed implementation of determining the mask of the first target is similar to and can refer to the aforementioned implementation of determining the mask of each of the targets in the designated target set, which is not repeated herein. Accordingly, the detailed implementation of determining the position of the first target in this embodiment is similar to and can refer to the aforementioned implementation of determining the position of each of the targets in the designated target set within the designated target volume, which is not repeated herein; accordingly, the detailed implementation of determining the dose of the first target is similar to and can refer to the aforementioned implementation of determining the dose of each of the targets in the designated target set in above process S104, which is not repeated herein.

In some embodiments, upon determining the position of the first target and the dose of the first target, the state matrix corresponding to the target volume is updated based on the dose of the first target.

In process S503, sizes, positions, and the doses of subsequent targets are sequentially determined based on the updated state matrix until the predetermined prescription dose is satisfied.

Based on the updated state matrix, the process of determining the size, position, and dose of the target is re-executed, and the state matrix is updated every time a size, position, and dose of one target are determined, until the dose distribution of the targets determined for the target volume corresponding to the target volume contour meets the requirements of the predetermined prescription dose and the number of targets does not exceed the predetermined threshold for the number of targets in a single target volume. In some embodiments, the dose distribution of the targets determined for the target volume corresponding to the target volume contour meeting the requirements of the predetermined prescription dose means that the sum of the total doses of all the targets determined for the target volume corresponding to the target volume contour reaches the predetermined prescription dose.

In some embodiments, the detailed implementation of determining the sizes, positions, and doses of the subsequent targets based on the updated state matrix is similar to and can refer to the aforementioned implementation of determining the size, position, and dose of the first target based on the state matrix.

In some embodiments, on the basis of the method provided by the above embodiments, the state matrix further includes, in addition to the target volume mask, a dose state corresponding to the target volume. Accordingly, in some embodiments, updating the state matrix corresponding to the target volume in the above method includes:
calculating dose state information corresponding to t targets within the target volume based on a dose of a t^{th} target and a size of the target volume; wherein t is an integer greater than or equal to 1; and
updating the dose state in the state matrix based on the dose state information of the t targets.

The dose state information corresponding to the t targets within the target volume refers to the dose state information of the corresponding target volume with t targets being placed within the corresponding target volume. In other words, every time a dose of a target is determined using the above method, the dose state information of all current targets within the corresponding target volume is calculated based on the currently determined dose of the target and the size of the corresponding target volume, and thus the dose state is updated. The size of the target volume refers to a size of the three-dimensional region corresponding to the target volume. In some embodiments, in the case that t is 1, i.e., the first target, upon determining the dose of the first target, the dose state information of 1 target within the corresponding target volume is calculated based on the dose of the first target and the size of the corresponding target, and then the dose state in the state matrix is updated based on the dose state information of the 1 target. In the case that t is an integer greater than 1, upon determining the dose of the t^{th} target, the dose state information of the t targets within the corresponding target volume is calculated based on the dose of the t^{th} target, the size of the corresponding target, and the doses of the t−1 targets before the t^{th} target, and then the dose state in the state matrix is updated based on the dose state information of the t targets.

In some embodiments, the dose state as described above includes a dose coverage distribution, a dose conformity distribution, and a dose overflow distribution. The dose coverage distribution, the dose conformity distribution, and the dose overflow distribution are respectively one layer of a three-layer feature matrix in the state matrix. That is, the state matrix includes a four-layer feature matrix, wherein the four layers of the feature matrix respectively are a target volume mask, a dose coverage distribution, a dose conformity distribution, and a dose overflow distribution. In some embodiments, the target volume mask is a first layer of the feature matrix, the dose coverage distribution is a second layer of the feature matrix, the dose conformity distribution is a third layer of the feature matrix, and the dose overflow distribution is a fourth layer of the feature matrix. In some embodiments, the dose coverage distribution is configured to characterize a dose-distributed region within the corresponding target volume, the dose conformity distribution is configured to characterize a underdosage region within the corresponding target volume, and the dose overflow distribution is configured to characterize a dose overflow region within the corresponding target volume.

Accordingly, the dose state information includes dose coverage information, dose conformity information, and dose overflow information.

In some embodiments, the dose coverage information, the dose conformity information, and the dose overflow information of the t targets in the corresponding target volume are calculated based on the dose of the t^{th} target and the size of the corresponding target volume respectively using the following Formula (1) to Formula (3).

$$C o {v}_{t} = \frac{{dose}_{50 \%}^{t} * PTV}{PTV} \qquad Formula ( 1 )$$

$$Co {n}_{t} = \frac{{dose}_{50 \%}^{t}}{PTV} \qquad Formula ( 2 )$$

$${Out}_{t} = \frac{{dose}_{50 \%}^{t} - {dose}_{50 \%}^{t} * PTV}{PTV} \qquad Formula ( 3 )$$

Wherein Covₜ represents the dose coverage information of t targets, which is also known as dose coverage degree,

$${dose}_{50 \%}^{t}$$

represents the 50% doses of the t^{th} target as well as the t−1^{st} target, i.e., 50% dose upon the front and back targets being calculated, PTV represents the size of the corresponding target volume, Conₜ represents the dose conformity information of the t targets, which is also known as the dose conformity degree, and Outₜ represents the dose overflow information of the t targets, which is also known as the dose overflow degree.

In some embodiments, in the method described above, updating the dose state based on the dose state information of the t targets includes:
updating the dose coverage distribution, the dose conformity distribution, and the dose overflow distribution respectively based on the dose coverage information, the dose conformity information, and the dose overflow information of the t targets.

That is, the dose coverage distribution in the state matrix is updated based on the dose coverage information of the t targets; the dose conformity distribution in the state matrix is updated based on the dose conformity information of the t targets; and the dose overflow distribution in the state matrix is updated based on the dose overflow information of the t targets.

In process S504, a number of the above targets is counted, and the number of the above targets and the size of each of the targets are determined as the target set within the contour.

In some embodiments, in the case that the total dose of all the targets determined for the target volume corresponding to the target volume contour satisfies the predetermined prescription dose, the numbers of targets of different sizes among all the targets determined for the target volume corresponding to the target volume contour are counted to acquire the numbers of targets of different sizes corresponding to the target volume contour, and the number of targets and the sizes of the various targets are stored as a target set within the target volume contour. That is, in some embodiments, the number of targets in the target set within the target volume contour includes the numbers of targets with different sizes. In some embodiments, the target set within the target volume contour includes X targets of size A, Y targets of size B, . . . and so on.

Of course, in some other embodiments, the number of targets is the total number of targets in the target volume corresponding to the target volume contour, and the sizes of the targets are listed target by target, such as the first target of size A, the second target of size B . . . the t^{th} target of size U, . . . and so on.

In some embodiments, on the basis of the method described in the above embodiments, the method further includes:

calculating reward information of the t^{th} target based on the dose coverage information, the dose conformity information, and the dose overflow information of the t targets.

In some embodiments, the reward information of the t^{th} target is calculated based on the dose coverage information, the dose conformity information, and the dose overflow information of the t targets and the dose coverage information, the dose conformity information, and the dose overflow information for the t−1 targets using the following Formula (4).

$${R}_{t} = ( C o {v}_{t} - C o {v}_{{t}_{t - 1}} ) + ( C o {n}_{t} - C o {n}_{t - 1} ) - ( {Out}_{t} - {Out}_{t - 1} ) - 0.5 \qquad Formula ( 4 )$$

Wherein, Rₜ represents the reward information of the t^{th} target, Covₜ represents the dose coverage information of the t targets,

$$C o {v}_{{t}_{t - 1}}$$

represents the dose coverage information of the t−1 targets, Conₜ represents the dose conformity information of the t targets, Conₜ₋₁ represents the dose conformity information of the t−1 targets, Outₜ represents the dose overflow information of the t targets, and Outₜ₋₁ represents the dose overflow information of the t−1 targets.

In some embodiments, the method further includes:
calculating current cumulative reward information corresponding to the t targets within the target volume based on the reward information for the t^{th} target; wherein the current cumulative reward information indicates the reliability of the current target set within the contour.

In some embodiments, the current cumulative reward information of the t targets within the corresponding target volume is acquired by accumulating the reward information of each target using the following Formula (5) based on a reward discount rate corresponding to each target in the t targets.

$${R}_{i} = {\sum }_{t = 1}^{T} {γ}^{t - 1} {R}_{t} \qquad Formula ( 5 )$$

Wherein, Rᵢ represents the current cumulative reward information of an i^{th} planning strategy πᵢ for the corresponding target volume, where i represents the current planning strategy, which means that one planning strategy for the corresponding target volume is acquired every time the size, position, and dose of one target is calculated; and γ^{t-1} represents the reward discount rate corresponding to the t−1^{st} target, where γ is calculated based on the predetermined maximum number of targets (which is also known as the target number threshold). In some embodiments, γ is approximated as 0.1^{1/20}≈0.9, which is configured to characterize the effect of 20 steps of action behind the t targets on the current state. Due to the presence of the discount rate, the targets added and determined by later actions have less impact on the target determined by the current action. Rₜ represents the reward information of the t^{th} target, wherein t is an integer greater than or equal to 1.

The cumulative reward information is the cumulative reward information of the t targets. The higher the value of the cumulative reward information is, the higher the reliability of the target set within the target volume contour is, and conversely, the lower the value of the cumulative reward information is, the lower the reliability of the current target set within the target volume contour is.

In some embodiments, the method further includes:
storing the updated state matrix corresponding to the target volume, the current target set within the target volume contour, and the current cumulative reward information corresponding to the target volume.

The updated state matrix, the current target set within the target volume contour, and the current cumulative reward information corresponding to the target volume are stored in a memory unit.

In some embodiments, the method further includes:
calculating a relative advantage parameter between the current target set within the contour and a history target set within the contour based on the current cumulative reward information and history cumulative reward information corresponding to the target volume, wherein the history cumulative reward information indicates a reliability of the history target set; and
determining and updating a target set within the target volume contour from the current target set and the history target set based on the relative advantage parameter.

In some embodiments, the relative advantage parameter between the current target set within the target volume contour and the history target set within the target volume contour, i.e., between the old and the new strategies of the target volume contour, is calculated using the following Formula (6) based on the current cumulative reward information and the history cumulative reward information corresponding to the target volume.

$${J}_{P P O 2}^{{θ}^{k}} ( θ ) \approx {\sum }_{( {s}_{t} , {a}_{t} )} min ( \frac{{p}_{θ} ( {a}_{t} \vert {s}_{t} )}{{p}_{{θ}^{k}} ( {a}_{t} \vert {s}_{t} )} {A}^{{θ}^{k}} ( {s}_{t} , {a}_{t} ) , clip ( \frac{{p}_{θ} ( {a}_{t} \vert {s}_{t} )}{{p}_{{θ}^{k}} ( {a}_{t} \vert {s}_{t} )} , 1 - ε , 1 + ε ) {A}^{{θ}^{k}} ( {s}_{t} , {a}_{t} ) ) \qquad Formula ( 6 )$$

In the above formula,

$$\frac{{P}_{θ} ( {a}_{t} \vert {s}_{t} )}{{P}_{{θ}^{k}} ( {a}_{t} \vert {s}_{t} )}$$

represents the ratio of the old and new strategies employing the action aₜ under the state of sₜ. In this embodiment, the action aₜ represents the currently determined target size, P_{θ}(aₜ|sₜ) represents the probability of selecting the history target set under the action aₜ, and P_{θ}_{^{k}}(aₜ|sₜ) represents the probability of selecting the current target set under the action at. Therefore, the ratio of old and new strategies is actually the ratio of the probability of selecting the current target set and the probability of selecting the history target set under sₜ state. The probability of selecting the current target set is determined and acquired based on the current cumulative reward information, and the probability of selecting the history target set is determined and acquired based on the history cumulative reward information. A^{θ}^{^{k}} represents the expectation of the reward under the current strategy, which is calculated based on the current cumulative reward information and the probability of selecting the current target set, and & represents a predetermined clipping coefficient of the advantage function, and

$${J}_{P P O 2}^{{θ}^{k}}$$

represents the relative advantage parameter.

In some embodiments, upon calculating and acquiring the relative advantage parameter, an optimal target set is determined from the current target set and the history target set based on the relative advantage parameter, and the target set within the target volume contour in the memory unit is updated to the optimal target set. In some embodiments, in the case that the relative advantage parameter satisfies a first condition, the current target set is determined to be superior to the history target set, and thus the current target set is determined and updated as the target set within the target volume contour; and in the case that the relative advantage parameter satisfies a second condition, the history target set is determined to be superior to the current target set, and the history target set is determined to be the optimal target set within the target volume contour.

According to the method provided by the embodiments, the target set corresponding to the target volume contour is acquired through deep reinforcement learning and training on the target volume contour, which achieves repeated trial and error by a machine during the deep reinforcement learning and training process, avoids manually setting the target combinations, eliminates the need for human experience data as a training sample, greatly reduces the human labor cost, at the same time reduces the reliance on clinical experience, ensures the accuracy of the target set within the target volume contour, and then effectively ensures the precision as well as the generation efficiency of the treatment plan.

In order to better understand the technical solutions provided by the present disclosure, the deep reinforcement learning and training process of the target volume contour is explained and illustrated hereafter by some detailed embodiments in conjunction with the accompanying drawings. FIG. 6 is a schematic diagram of a framework of a training module in a method for generating a treatment plan according to some embodiments of the present disclosure, wherein the training module is an algorithmic model for performing deep reinforcement learning and training on target volume contours. In some embodiments, the training module is configured to train a plan model that meets the requirements of a predetermined prescription dose, i.e., a target set within the target volume contour, based on the input target volume contour, which is also known as the target volume shape. The process of training the target volume contour using the training module is actually an experience accumulation process of a machine through trial and error, which is equivalent to the trial and error process of a physicist when forming a treatment plan.

The input of the training module is a target volume mask of a target volume corresponding to a target volume contour formed based on the target volume contour.

State features of the environment are divided into four parts, accordingly, the state matrix is a four-layer feature matrix with the first layer of the feature matrix, i.e., State [0], is the target volume mask, the second layer of the feature matrix, i.e., State [1], is the dose coverage distribution, the third layer of the feature matrix, i.e., State [2], is the dose conformity distribution, and the fourth layer of the feature matrix, i.e., State [3], is the dose overflow distribution.

Agent of the deep reinforcement learning and training includes a CNN and two different networks connected by the CNN, i.e., actor network (ActorNet) and critic network (CriticNet). The CNN is configured to acquire a state feature by performing feature extraction on the state matrix; the actor network is configured to select an action based on the state feature, i.e., determine a size of a target within a target volume corresponding to the state feature based on the state feature; and the critic network is configured to calculate the state value parameter of the target volume corresponding to the state feature based on the state feature, i.e., acquire a state critic parameter value, then calculate a loss function value of the agent based on the state critic parameter value, and then determine whether the loss function value meets an iteration stopping condition.

$$\begin{matrix}L = α {L}_{actor} + β {L}_{critic} \\ {L}_{actor} = mean ( min ( ratio * {A}_{t} , clip ( ratio , 1 - ε , 1 + ε ) * {A}_{t} ) ) \\ {L}_{critic} = {( {A}_{t} + value - {V}_{critic} )}^{2}\end{matrix}$$

Wherein α=1, β=0.5, L_{actor} represents the loss function value of ActorNet, L_{critic} represents the loss function value of CriticNet. Radio represents the relative advantage of the old and new strategies, i.e., the relative advantage parameter, value represents the state critic parameter value, and Aₜ represents the selected action at moment t, i.e., the size of the target determined at moment t.

In some embodiments, the size of the target within the target volume contour is acquired by deep reinforcement learning and training on the target volume contour using the trained agent, and the position of each of the targets is determined by combination with the way of convolution shape matching.

The following describes the reward mechanism of deep reinforcement learning and training process. The reward is divided into two parts, that is, real-time reward and delayed reward, wherein the real-time reward means that the growth of the values of 50% dose coverage and the target volume conformity is calculated as a positive reward upon the sizes of a front and back targets being acquired, and at the same time, a negative reward is provided every time one target is placed on the premise of using a smallest possible total number of targets; and the delayed reward means that a larger positive reward for the dose coverage is provided in the case that the predetermined prescription dose is reached, and ultimately cumulative reward information (i.e., cumulative reward) for each planning strategy (i.e., each target) is calculated.

An experience memory unit is configured to store combination information of state matrix St, action At, reward Rt, value Vt, or the like. A detailed description of the state matrix St refers to the above descriptions, the action At is the size of the t^{th} target determined using the actor network, the reward Rt is the reward information of the t^{th} target as described above, and the value Vt is the critic parameter value acquired by criticizing the state matrix St.

A strategy updating module is configured to optimize and update the target set within the target volume contour (i.e., the strategy) stored in the memory unit using a proximal policy optimization (PPO) algorithm. In some embodiments, a relative advantage parameter between a current target set within a target volume contour and a history target set within the target volume contour (i.e., between the old and new strategies of the target volume contour) is calculated using the PPO algorithm based on the current cumulative reward information corresponding to a target volume and the history cumulative reward information corresponding to the target volume, wherein the current target set represents a new strategy (New_n) of the corresponding target volume, and the history target set represents an old strategy (Old_n) of the corresponding target volume.

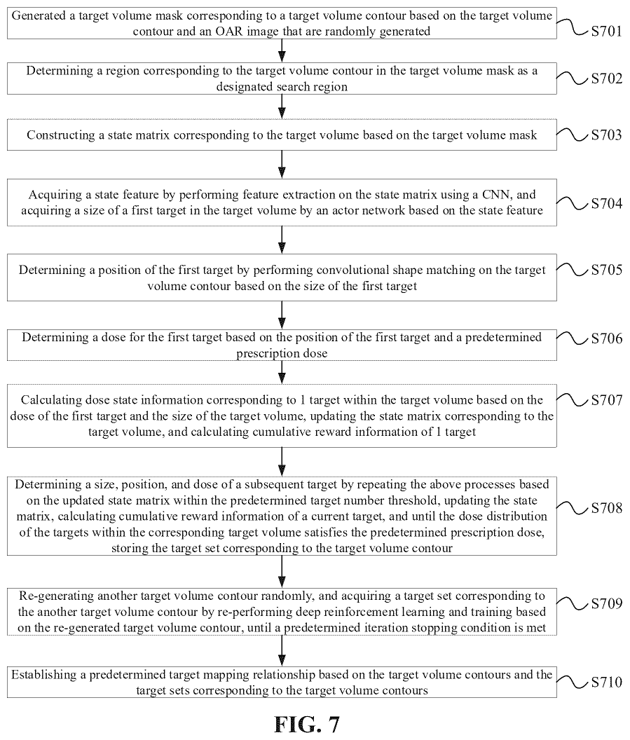

FIG. 7 is a flowchart of yet another method of deep reinforcement learning and training of target volume contours in a method for generating a treatment method according to some embodiments of the present disclosure. As shown in FIG. 7, the method includes the following processes.

In process S701, a target volume mask corresponding to a target volume contour is generated based on the target volume contour and an OAR image that are randomly generated.

In process S702, a region corresponding to the target volume contour in the target volume mask is determined as a designated search region.

By determining the region corresponding to the target volume contour in the target volume mask as the designated search region, the target position is restricted to the region corresponding to the target volume contour, avoiding the target position from falling into other regions, thereby avoiding unnecessary damage to other regions, such as the region of the organ at risk, during treatment, and thereby reducing the search space and improving the efficiency of training and plan generation.

In process S703, a state matrix corresponding to the target volume is constructed based on the target volume mask.

In process S704, a state feature is acquired by performing feature extraction on the state matrix using a CNN, and a size of a first target in the target volume is acquired by an actor network based on the state feature.

In process S705, a position of the first target is determined by performing convolutional shape matching on the target volume contour based on the size of the first target.

In process S706, a dose for the first target is determined based on the position of the first target and a predetermined prescription dose.

In process S707, dose state information corresponding to 1 target within the target volume is calculated based on the dose of the first target and the size of the target volume, the state matrix corresponding to the target volume is updated, and cumulative reward information of 1 target is calculated.

In process S708, a size, position, and dose of a subsequent target are determined by repeating the above processes based on the updated state matrix within the predetermined target number threshold, the state matrix is updated, cumulative reward information of a current target is calculated, and until the dose distribution of the targets within the corresponding target volume satisfies the predetermined prescription dose, the target set corresponding to the target volume contour is stored.

In process S709, another target volume contour is re-generated randomly, and a target set corresponding to the another target volume contour is acquired by re-performing deep reinforcement learning and training based on the re-generated target volume contour, until a predetermined iteration stopping condition is met.

In process S710, a predetermined target mapping relationship is established based on the target volume contours and the target sets corresponding to the target volume contours.

According to the method provided by the embodiments, the target set corresponding to the target volume contour is acquired through deep reinforcement learning and training on the target volume contours that are generated randomly, which achieves repeated trial and error by a machine during the deep reinforcement learning and training process, greatly reduces the human labor cost, reduces the reliance on clinical experience, ensures the accuracy of the target set within the target volume contour, and then effectively ensures the precision as well as the generation efficiency of the treatment plan.

The following describes an apparatus for generating a treatment plan, a device, and a storage medium provided by the present disclosure for performing the above methods, and the detailed implementation as well as the technical effect thereof can refer to the above descriptions, and are not repeated hereafter.

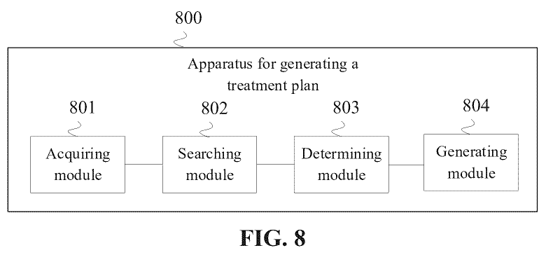

FIG. 8 is a schematic diagram of an apparatus for generating a treatment plan according to some embodiments of the present disclosure. As shown in FIG. 8, the apparatus 800 for generating a treatment plan includes:
an acquiring module 801, configured to acquire a designated contour of a designated target volume;
a searching module 802, configured to search, in a predetermined target mapping relationship, a designated target set corresponding to the designated contour, the designated target set comprising a total number of targets and a size of each of the targets;
a determining module 803, configured to determine a position of each of the targets within the designated target volume based on the size of each of the targets; and
a generating module 804, configured to determine a dose of each of the targets based on the position of each of the targets and a predetermined prescription dose, and generate a treatment plan.

In some embodiments, the determining module 803 is further configured to determine a mask of each of the targets based on the size of each of the targets; and determine the position of each of the targets within the designated target volume by performing convolutional shape matching between the mask and the designated contour.

In some embodiments, the generating module 804 is further configured to acquire a dose curve distribution in the designated target volume by performing a dose calculation based on the size, the position, and a weight of each of the targets; and determine the dose of each of the targets based on the dose curve distribution and the predetermined prescription dose.

In some embodiments, the apparatus 800 for generating a treatment plan further includes: a training module, configured to acquire a plurality of target volumes; acquire contours of the plurality of target volumes by delineating the plurality of target volumes; acquire target sets corresponding to the contours by deep reinforcement learning and training based on each of the contours; and establish the predetermined target mapping relationship based on the contours and the target sets corresponding to the contours.

In some embodiments, the training module is further configured to form, based on each of the contours, a mask of a target volume corresponding to the contour; construct a state matrix corresponding to the target volume based on the mask, wherein the state matrix includes the mask; and acquire a target set within the contour based on the state matrix.

In some embodiments, the training module is further configured to acquire a size of a first target by performing feature extraction on the state matrix based on a convolutional neural network; determine a position of the first target and a dose of the first target based on the size of the first target, and update the state matrix corresponding to the target volume; determine sizes, positions, and doses of subsequent targets sequentially based on the updated state matrix until the predetermined prescription dose is satisfied; and count a number of the targets and determine the number of the targets and the sizes of the targets as the target set within the contour.

In some embodiments, the training module is further configured to acquire an initial state feature corresponding to the target volume by performing feature extraction on the state matrix using the convolutional neural network; and acquire the size of the first target by processing the initial state feature using a predetermined action selection network.

In some embodiments, the training module is further configured to determine a mask of the first target based on the size of the first target; determine the position of the first target by performing convolutional shape matching between the contour and the mask of the first target; and determine the dose of the first target based on the position of the first target.

In some embodiments, the state matrix further includes a dose state corresponding to the target volume, and the training module is further configured to calculate dose state information corresponding to t targets within the target volume based on a dose of a t^{th} target and a size of the target volume, wherein t is an integer greater than or equal to 1; and update the dose state based on the dose state information of the t targets.

In some embodiments, the dose state includes a dose coverage distribution, a dose conformity distribution, and a dose overflow distribution; and the dose state information includes dose coverage information, dose conformity information, and dose overflow information.

The training module is further configured to update the dose coverage distribution, the dose conformity distribution, and the dose overflow distribution respectively based on the dose coverage information, dose conformity information, and dose overflow information of the t targets.

In some embodiments, the training module is further configured to calculate reward information of the t^{th} target based on the dose coverage information, dose conformity information, and dose overflow information of the t targets.

In some embodiments, the training module is further configured to calculate current cumulative reward information corresponding to the t targets within the target volume based on the reward information of the t^{th} target; wherein the current cumulative reward information indicates a reliability of a current target set within the contour.

In some embodiments, the training module is further configured to store the updated state matrix, the current target set within the target volume contour, and the current cumulative reward information.

In some embodiments, the training module is further configured to calculate a relative advantage parameter between the current target set within the contour and a previous target set within the contour based on the current cumulative reward information and history cumulative reward information corresponding to the target volume, wherein the history cumulative reward information indicates a reliability of the previous target set; and determine and update a designated target set within the contour from the current target set and the previous target set based on the relative advantage parameter.

The above apparatus is configured to perform the method for generating a treatment plan according to the foregoing embodiments, and the apparatus is similar in implementation principle and technical effect, which are not repeated herein.

In some embodiments, the above modules are one or more integrated circuits configured to perform the above methods, such as one or more application-specific integrated circuits (ASIC), one or more microprocessors (i.e., digital signal processor, DSP), one or more field-programmable gate arrays (FPGAs), or the like. In some embodiments, in the case that one of the above modules is implemented in the form of a processing element scheduling one or more program codes, the processing element is a general-purpose processor, such as a central processing unit (CPU), or other processors that are capable of invoking one or more program codes. Further, in some embodiments, the modules are integrated and implemented in the form of a system-on-a-chip (SOC).

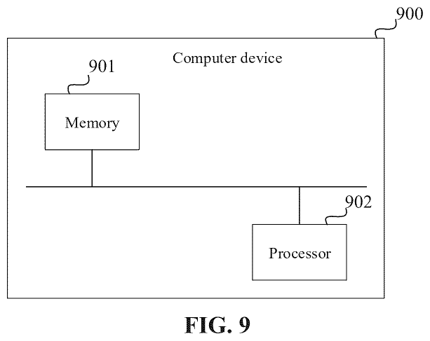

FIG. 9 is a schematic diagram of a computer device according to some embodiments of the present disclosure, and a specific product form of the computer device is a computing device or a server having computing processing functions in some embodiments.

The computer device 900 includes a memory 901 and a processor 902. The memory 901 and the processor 902 are connected via a bus.

The memory 901 stores one or more computer programs executable by the processor 902, and the processor 902, when loading and executing the one or more computer programs, is configured to perform the above method embodiments. The specific implementations and technical effects of the device are similar to the method embodiments and are not repeated herein.

On the basis of the above-described radiotherapy dose determination method, the embodiments of the present disclosure further provide a computer-readable storage medium for performing the above-described radiotherapy dose determination method. In some embodiments, the storage medium is a non-transitory storage medium storing one or more computer programs, wherein the computer programs, when loaded and executed by a processor of a device, cause the device to perform the above-described method embodiments.

Based on the several embodiments of the present disclosure, it is understood that the apparatus and methods disclosed above can be implemented in other ways. The apparatus embodiments described above are only illustrative, the division of the units described above is merely a logical and functional division, and the apparatus can be divided in other ways in actual implementation. In some embodiments, a plurality of units or components are combined or integrated into another system, or some of the features are omitted or not performed. Further, in some embodiments, the coupling, direct coupling, or communication connection between elements shown or discussed is an indirect coupling or communication connection through some interface, device, or unit, which is electrical, mechanical, or otherwise.

The units illustrated as separated components are or are not physically separated, and components shown as units are or are not physical units, i.e., located in a single place or distributed over a plurality of network units. Some or all of these units are selected to achieve the purpose of the technical solutions of the embodiments depending on actual needs.

In addition, the various functional units in the various embodiments of the present disclosure are integrated in a single processing unit, or physically exist separately; or two or more units are integrated in a single unit. The above integrated units are achieved either in the form of hardware or in the form of hardware plus software functional units.

In some embodiments, the above-described integrated units achieved in the form of software function units are stored in a computer-readable storage medium. The above-described software functional unit is stored in a storage medium and includes a plurality of instructions, wherein the plurality of instructions, when loaded and run by a processor of a computer device (which is a personal computer, a server, or a network device, etc.), cause the device or the processor to perform some of the processes of the methods described in various embodiments of the present disclosure. The aforementioned storage medium includes a USB flash disk, a portable hard disk, a read-only memory (ROM), a random access memory (RAM), a magnetic disk, a compact disc, or other media that can store one or more program codes.

The above are only some specific embodiments of the present disclosure, and the scope of protection of the present disclosure is not limited thereto. Any modifications or equivalent replacements made by any person skilled in the art within the scope of the technical solutions provided by the embodiments of the present disclosure should be included within the scope of protection of the present disclosure. Therefore, the protection scope of this application is subject to the claims.

1. A method for generating a treatment plan, comprising:
acquiring a designated contour of a designated target volume;
searching, in a predetermined target mapping relationship, a designated target set corresponding to the designated contour, the designated target set comprising a total number of targets and a size of each of the targets;
determining a position of each of the targets within the designated target volume based on the size of each of the targets; and
determining a dose of each of the targets based on the position of each of the targets and a predetermined prescription dose, and generating a treatment plan.

2. The method according to claim 1, wherein determining the position of each of the targets within the designated target volume based on the size of each of the targets comprises:
determining a mask of each of the targets based on the size of each of the targets; and
determining the position of each of the targets within the designated target volume by performing convolutional shape matching between the mask and the designated contour.

3. The method according to claim 1, wherein determining the dose of each of the targets based on the position of each of the targets and the predetermined prescription dose comprises:
acquiring a dose curve distribution in the designated target volume by performing a dose calculation based on the size, the position, and a weight of each of the targets; and
determining the dose of each of the targets based on the dose curve distribution and the predetermined prescription dose.

4. The method according to claim 1, wherein prior to searching, in the predetermined target mapping relationship, the designated target set corresponding to the designated contour, the method further comprises:
acquiring a plurality of target volumes;
acquiring contours of the plurality of target volumes by delineating the plurality of target volumes;
acquiring target sets corresponding to the contours by deep reinforcement learning and training based on each of the contours; and
establishing the predetermined target mapping relationship based on the contours and the target sets corresponding to the contours.

5. The method according to claim 4, wherein acquiring the target sets corresponding to the contours by deep reinforcement learning and training based on each of the contours comprises:
forming, based on each of the contours, a mask of a target volume corresponding to the contour;
constructing a state matrix corresponding to the target volume based on the mask, wherein the state matrix comprises the mask; and
acquiring a target set within the target volume based on the state matrix.

6. The method according to claim 5, wherein acquiring the target set within the target volume based on the state matrix comprises:
acquiring a size of a first target by performing feature extraction on the state matrix based on a convolutional neural network;
determining a position of the first target and a dose of the first target based on the size of the first target, and updating the state matrix corresponding to the target volume;
determining sizes, positions, and doses of subsequent targets sequentially based on the updated state matrix until the predetermined prescription dose is satisfied; and
counting a number of the targets and determining the number of the targets and the sizes of the targets as the target set within the target volume.

7. The method according to claim 6, wherein acquiring the size of the first target by performing feature extraction on the state matrix based on the convolutional neural network comprises:
acquiring an initial state feature corresponding to the target volume by performing feature extraction on the state matrix using the convolutional neural network; and
acquiring the size of the first target by processing the initial state feature using a predetermined action selection network.

8. The method according to claim 6, wherein determining the position of the first target and the dose of the first target based on the size of the first target comprises:
determining a mask of the first target based on the size of the first target;
determining the position of the first target by performing convolutional shape matching between the contour and the mask of the first target; and
determining the dose of the first target based on the position of the first target.

9. The method according to claim 6, wherein the state matrix further comprises a dose state corresponding to the target volume, and updating the state matrix corresponding to the target volume comprises:
calculating dose state information corresponding to t targets within the target volume based on a dose of a t^{th} target and a size of the target volume, wherein t is an integer greater than or equal to 1; and
updating the dose state based on the dose state information of the t targets.

10. The method according to claim 9, wherein the dose state comprises a dose coverage distribution, a dose conformity distribution, and a dose overflow distribution; and the dose state information comprises dose coverage information, dose conformity information, and dose overflow information; and
updating the dose state based on the dose state information of the t targets comprises:
updating the dose coverage distribution, the dose conformity distribution, and the dose overflow distribution respectively based on the dose coverage information, dose conformity information, and dose overflow information of the t targets.

11. The method according to 10, further comprising:
calculating reward information of the t^{th} target based on the dose coverage information, dose conformity information, and dose overflow information of the t targets.

12. The method according to claim 11, further comprising:
calculating current cumulative reward information corresponding to the t targets within the target volume based on the reward information of the t^{th} target;
wherein the current cumulative reward information indicates a reliability of a current target set within the target volume.

13. The method according to 12, further comprising:
storing the updated state matrix, the current target set within the target volume, and the current cumulative reward information.

14. The method according to claim 13, further comprising:
calculating a relative advantage parameter between the current target set within the target volume and a previous target set within the target volume based on the current cumulative reward information and history cumulative reward information corresponding to the target volume, wherein the history cumulative reward information indicates a reliability of the previous target set; and
determining and updating a designated target set within the target volume from the current target set and the previous target set based on the relative advantage parameter.

15. (canceled)

16. A computer device for generating a treatment plan, comprising: a memory and a processor, wherein the memory stores one or more computer programs executable by the processor, and the processor, when loading and executing the one or more computer programs, is caused to:
acquire a designated contour of a designated target volume;
search, in a predetermined target mapping relationship, a designated target set corresponding to the designated contour, the designated target set comprising a total number of targets and a size of each of the targets;
determine a position of each of the targets within the designated target volume based on the size of each of the targets; and
determine a dose of each of the targets based on the position of each of the targets and a predetermined prescription dose, and generating a treatment plan.

17. A non-transitory storage medium, storing one or more computer programs, wherein the one or more computer programs, when read and run by a processor of a device, cause the device to:
acquire a designated contour of a designated target volume;
search, in a predetermined target mapping relationship, a designated target set corresponding to the designated contour, the designated target set comprising a total number of targets and a size of each of the targets;
determine a position of each of the targets within the designated target volume based on the size of each of the targets; and
determine a dose of each of the targets based on the position of each of the targets and a predetermined prescription dose, and generating a treatment plan.

18. The computer device according to claim 16, wherein the processor, when loading and executing the one or more computer programs, is caused to:
determine a mask of each of the targets based on the size of each of the targets; and
determine the position of each of the targets within the designated target volume by performing convolutional shape matching between the mask and the designated contour.

19. The computer device according to claim 16, wherein the processor, when loading and executing the one or more computer programs, is caused to:
acquire a dose curve distribution in the designated target volume by performing a dose calculation based on the size, the position, and a weight of each of the targets; and
determine the dose of each of the targets based on the dose curve distribution and the predetermined prescription dose.

20. The computer device according to claim 16, wherein the processor, when loading and executing the one or more computer programs, is caused to:
acquire a plurality of target volumes;
acquire contours of the plurality of target volumes by delineating the plurality of target volumes;
acquire target sets corresponding to the contours by deep reinforcement learning and training based on each of the contours; and
establish the predetermined target mapping relationship based on the contours and the target sets corresponding to the contours.

21. The computer device according to claim 20, wherein the processor, when loading and executing the one or more computer programs, is caused to:
form, based on each of the contours, a mask of a target volume corresponding to the contour;
construct a state matrix corresponding to the target volume based on the mask, wherein the state matrix comprises the mask; and
acquire a target set within the target volume based on the state matrix.
